(19) United States
(12) Patent Application Publication
(10) Pub. No.: US 2026/0237477 A1
(43) Pub. Date: Aug. 13, 2026

(54) SYSTEM AND METHOD FOR AUTOMATED FORMATTING OF MEDICAL DATA IN REMOTE INTERVENTIONS

(71) Applicant: UpDoc Inc., Los Altos, CA (US)

(72) Inventors: Sharif Vakili, Los Altos, CA (US); Ashwin K. Nayak, Mountain View, CA (US)

(21) Appl. No.: 19/048,790

(22) Filed: Feb. 7, 2025

(51) Int. Cl. G16H 15/00; G10L 15/26; G16H 10/60; G16H 40/67

(52) U.S. Cl. CPC G16H 15/00; G10L 15/26; G16H 10/60; G16H 40/67

(57) ABSTRACT

A system and method for processing conversational data from a patient interaction with a conversational AI agent to automatically generate a clinical progress note for the interaction. In accordance with certain aspects of the present disclosure, a conversational interaction with a patient and a conversational AI agent may be facilitated in accordance with a remote intervention protocol for one or more medical conditions. Conversational data generated during the conversational interaction may be processed according to at least one data processing framework. The conversational data and other medical data for the patient may be processed by a generative AI engine to automatically generate a progress note for medical billing for the conversational interaction.

### FIELD

The present disclosure relates to the field of generative artificial intelligence (AI) technology for use in medical data processing; in particular, a system and method for processing conversational data from a patient interaction with a conversational AI agent to automatically generate a clinical progress note for the interaction.

### BACKGROUND

The healthcare industry is burdened by extensive administrative requirements, including the creation of clinical progress notes essential for accurate medical billing. These notes document patient encounters, ensuring compliance with regulatory standards, payer requirements, and quality care metrics. Traditionally, this process relies on manual or semi-automated methods, requiring healthcare providers to dictate, transcribe, or manually input data into electronic health record (EHR) systems. This approach is time-consuming, error-prone, and often detracts from the provider's focus on patient care.

Advances in artificial intelligence (AI), particularly in the domain of generative AI, present opportunities to revolutionize clinical documentation processes. Generative AI models, such as those leveraging natural language processing (NLP) and deep learning, can synthesize and generate human-like text based on structured and unstructured inputs. These models have shown promise in understanding medical terminologies, interpreting context, and generating coherent, domain-specific text.

Existing solutions for clinical documentation often lack the ability to seamlessly integrate patient-specific data from diverse sources, such as EHR systems, voice inputs, or real-time monitoring devices, into a cohesive progress note. Furthermore, many current systems struggle to ensure compliance with evolving regulatory and billing standards, necessitating additional manual interventions to verify the accuracy and completeness of generated notes.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Certain aspects of the present disclosure provide for a method for automatically formatting medical data for remote patient interventions. In accordance with certain embodiments, the method may comprise one or more steps or operations for configuring (e.g., with at least one server) a remote intervention protocol for a patient. The method may further comprise one or more steps or operations for configuring (e.g., with the at least one server) a generative artificial intelligence (AI) model according to the remote intervention protocol for the patient. The method may further comprise one or more steps or operations for instantiating (e.g., with the at least one server) a conversational interaction between a conversational AI agent and a first client device associated with the patient (e.g., wherein the first client device comprises an audio input/output means). The method may further comprise one or more steps or operations for outputting (e.g., with the conversational AI agent) one or more generative prompts according to the generative AI model at the first client device. The method may further comprise one or more steps or operations for receiving (e.g., with the at least one server) one or more conversational responses from the patient via the first client device. The method may further comprise one or more steps or operations for converting (e.g., with the at least one server) the one or more conversational responses from an audio data format to a text data format (e.g., wherein the text data format comprises a transcript of the conversational interaction between the conversational AI agent and the patient). The method may further comprise one or more steps or operations for processing (e.g., with the at least one server) the transcript of the conversational interaction and one or more electronic medical records for the patient according to the generative AI model to generate a clinical note for the patient. The method may further comprise one or more steps or operations for storing (e.g., in a non-transitory storage device communicably engaged with the at least one server) the clinical note for the patient in a standardized format. The method may further comprise one or more steps or operations for providing (e.g., with the at least one server) a notification to a second client device associated with a provider user in response to storing the clinical note. The method may further comprise one or more steps or operations for rendering (e.g., with the at least one server) the clinical note in the standardized format at the second client device.

In accordance with certain embodiments, the method for automatically formatting medical data for remote patient interventions may include one or more steps or operations for receiving (e.g., at a user interface of the second client device) an electronic signature for the provider user in response to rendering the clinical note. In certain embodiments, the method may further comprise one or more steps or operations for applying (e.g., with the at least one server) the electronic signature for the provider user to the clinical note and storing the signed clinical note in the standardized format. In certain embodiments, the standardized format may comprise an electronic data interchange format for at least one electronic medical billing system. In certain embodiments, the method may further comprise one or more steps or operations for communicating (e.g., with the at least one server) the signed clinical note to at least one third-party server via an electronic data interchange. In certain embodiments, the method may further comprise one or more steps or operations for initiating (e.g., with the at least one server) at least one routine of the remote intervention protocol in response to at least one user-generated input by the patient at the first client device. In certain embodiments, the method may further comprise one or more steps or operations for processing (e.g., with the at least one server) the transcript of the conversational interaction and the one or more electronic medical records for the patient to determine one or more medical billing code for the conversational interaction. In certain embodiments, the method may further comprise one or more steps or operations for assigning (e.g., with the at least one server) the one or more determined medical billing code for the conversational interaction to the clinical note according to the standardized format. In certain embodiments, the method may further comprise one or more steps or operations for receiving (e.g., via the second client device) one or more user-generated inputs from the provider user (e.g., wherein the one or more user-generated inputs comprise one or more additions or revisions to the clinical note). In certain embodiments, the method may further comprise one or more steps or operations for updating (e.g., with the at least one server) the clinical note according to the one or more user-generated inputs from the provider user and storing the updated clinical note in the non-transitory storage device.

Further aspects of the present disclosure provide for a system automatically formatting medical data for remote patient interventions. In accordance with certain aspects of the present disclosure, the system may comprise a first client device (e.g., comprising an audio input/output means) associated with a patient user; a second client device (e.g., comprising a user interface) associated with a practitioner user; and at least one server communicably engaged with the first client device and the second client device via a network communications interface. In accordance with certain embodiments, the at least one server may comprise at least one processor and a non-transitory computer-readable storage device communicably engaged with the at least one processor. In accordance with said embodiments, the non-transitory computer-readable storage device may comprise a processor-executable instructions stored thereon that, when executed, cause the at least one processor to perform one or more operations. In accordance with certain aspects of the present disclosure, the one or more operations may comprise one or more steps or operations for configuring a remote intervention protocol for the patient user. In certain embodiments, the one or more operations may further comprise one or more steps or operations for configuring a generative artificial intelligence (AI) model according to the remote intervention protocol for the patient user. In certain embodiments, the one or more operations may further comprise one or more steps or operations for instantiating a conversational interaction between a conversational AI agent and the first client device. In certain embodiments, the one or more operations may further comprise one or more steps or operations for outputting one or more generative prompts according to the generative AI model at the first client device. In certain embodiments, the one or more operations may further comprise one or more steps or operations for receiving one or more conversational responses from the patient via the first client device. In certain embodiments, the one or more operations may further comprise one or more steps or operations for converting the one or more conversational responses from an audio data format to a text data format. In certain embodiments, the text data format may comprise a transcript of the conversational interaction between the conversational AI agent and the patient user. In certain embodiments, the one or more operations may further comprise one or more steps or operations for processing the transcript of the conversational interaction and one or more electronic medical records for the patient user according to the generative AI model to generate a clinical note for the patient user. In certain embodiments, the one or more operations may further comprise one or more steps or operations for storing (e.g., in the non-transitory computer-readable storage device) the clinical note for the patient user in a standardized format. In certain embodiments, the one or more operations may further comprise one or more steps or operations for providing (e.g., via the communications network) a notification to the second client device in response to storing the clinical note. In certain embodiments, the one or more operations may further comprise one or more steps or operations for rendering the clinical note in the standardized format at the second client device.

In accordance with certain embodiments of the system automatically formatting medical data for remote patient interventions, the one or more operations may further comprise operations for receiving (e.g., via the second client device) an electronic signature from the provider user in response to rendering the clinical note at the second client device. In certain embodiments, the one or more operations may further comprise operations for applying the electronic signature for the provider user to the clinical note and storing the signed clinical note in the standardized format. In certain embodiments, the standardized format comprises an electronic data interchange format for at least one electronic medical billing system. In said embodiments, the one or more operations may further comprise operations for communicating the signed clinical note to at least one third-party server via an electronic data interchange. In certain embodiments, the one or more operations may further comprise operations for processing the transcript of the conversational interaction and the one or more electronic medical records for the patient to determine one or more medical billing code for the conversational interaction. In said embodiments, the one or more operations may further comprise operations for assigning the one or more determined medical billing code for the conversational interaction to the clinical note according to the standardized format. In certain embodiments, the one or more operations may further comprise operations for receiving (e.g., via the second client device) one or more user-generated inputs from the provider user (e.g., wherein the one or more user-generated inputs comprise one or more additions or revisions to the clinical note). In said embodiments, the one or more operations may further comprise operations for updating the clinical note according to the one or more user-generated inputs from the provider user and storing the updated clinical note in the non-transitory storage device.

Still further aspects of the present disclosure provide for a non-transitory computer readable medium having processor-executable instructions stored thereon that, when executed by at least one processor, are configured cause the at least one processor to execute one or more operations of a method for automatically formatting medical data for remote patient interventions. In accordance with certain aspects of the present disclosure, the one or more operations may comprise one or more steps or operations for configuring a remote intervention protocol for the patient user; configuring a generative artificial intelligence (AI) model according to the remote intervention protocol for the patient user; instantiating a conversational interaction between a conversational AI agent and a first client device associated with the patient user; outputting one or more generative prompts according to the generative AI model at the first client device; receiving one or more conversational responses from the patient via the first client device; converting the one or more conversational responses from an audio data format to a text data format (e.g., wherein the text data format comprises a transcript of the conversational interaction between the conversational AI agent and the patient user); processing the transcript of the conversational interaction and one or more electronic medical records for the patient user according to the generative AI model to generate a clinical note for the patient user; storing (e.g., in the non-transitory computer-readable storage device) the clinical note for the patient user in a standardized format; providing (e.g., via the communications network) a notification to a second client device associated with a provider user in response to storing the clinical note; and rendering the clinical note in the standardized format at the second client device.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 is an architecture diagram of a generative AI medical documentation system, in accordance with certain aspects of the present disclosure;

FIG. 2 is a system diagram of a generative AI medical documentation system, in accordance with certain aspects of the present disclosure;

FIG. 3 is a process flow diagram of a routine of a generative AI medical documentation system, in accordance with certain aspects of the present disclosure;

FIG. 4 is a process flow diagram of a routine of a generative AI medical documentation system, in accordance with certain aspects of the present disclosure;

FIG. 5 is a process flow diagram of a routine of a generative AI medical documentation system, in accordance with certain aspects of the present disclosure;

FIG. 6 is a process flow diagram of a routine of a generative AI medical documentation system, in accordance with certain aspects of the present disclosure;

FIG. 7 is a process flow diagram of a routine of a generative AI medical documentation system, in accordance with certain aspects of the present disclosure;

FIG. 8 is a process flow diagram of a routine of a generative AI medical documentation system, in accordance with certain aspects of the present disclosure;

FIG. 9 is a process flow diagram of a generative AI medical billing method, in accordance with certain aspects of the present disclosure;

FIGS. 10A-10B are illustrations of an AI-generated clinical note, in accordance with certain aspects of the present disclosure;

FIG. 11 is a block diagram illustrating a representative software architecture, which may be used in conjunction with various hardware architectures herein described; and

FIG. 12 is a block diagram illustrating components of a machine, according to some exemplary embodiments, able to read instructions from a machine-readable medium (e.g., a machine-readable storage medium) and perform any one or more of the methodologies discussed herein.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, apparatuses and systems configured to receive, process and format multiple data types and data sources to automatically generate a clinical note for a remote patient interaction with a conversational AI agent.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Before the present invention and specific exemplary embodiments of the invention are described, it is to be understood that this invention is not limited to the particular embodiments described, and as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

As used herein, “exemplary” means serving as an example or illustration and does not necessarily denote ideal or best.

As used herein, the term “includes” means includes but is not limited to, the term “including” means including but not limited to. The term “based on” means based at least in part on.

As used herein, the term “interface” refers to any shared boundary across which two or more separate components of a computer system may exchange information. The exchange can be between software, computer hardware, peripheral devices, humans, and combinations thereof.

As used herein, the terms “computer,” “processor” and “computer processor” encompass a personal computer, a workstation computer, a tablet computer, a smart phone, a microcontroller, a microprocessor, a field programmable object array (FPOA), a digital signal processor (DSP), an application-specific integrated circuit (ASIC), a field programmable gate array (FPGA), a programmable logic array (PLA), or any other digital processing engine, device or equivalent capable of executing software code including related memory devices, transmission devices, pointing devices, input/output devices, displays and equivalents.

As used herein, the terms “application” or “app” refer to a software program, or a collection of software programs, that comprise a collection of processor-executable instructions that, when executed, are configured to perform at least one specific task for a user.

As used herein, the terms “conversational agent” or “conversational AI agent” or “agent” refer to any device, system and/or program configured to autonomously execute one or more objective function in response to one or more inputs. Said terms may be used interchangeably. The one or more inputs may comprise one or more user-generated inputs, sensor-based inputs, internal system inputs, external system inputs, environmental percepts, and the like. Examples of conversational agents may include, but are not limited to, one or more virtual assistant, personal assistant, chatbot, interactive virtual response (IVR) agent, and the like.

As used herein, the term “instance” means a case or occurrence of one or more executable operations of a software application including, but not limited to, a session, a graphical element, a document type, or a document that conforms to a particular data type definition.

As used herein, the term “mobile device” or “mobile electronic device” or “mobile computing device” includes any portable electronic device capable of executing one or more digital functions or operations; including, but not limited to, smart phones, tablet computers, personal digital assistants, wearable activity trackers, smart watches, smart speakers, and the like.

As used herein, a “portal” makes network resources (applications, databases, etc.) available to end users. The user can access the portal via a web browser, smart phone, tablet computer, and other client computing devices. Portals may include network enabling services such as e-mail, chat rooms and calendars that interact seamlessly with other applications.

As used herein, the term “transmit” and its conjugates means transmission of digital and/or analog signal information by electronic transmission, Wi-Fi, BLUETOOTH technology, wireless, wired, or other known transmission technologies including transmission to an Internet website.

As used herein, the terms “provider” and “practitioner” refer to a healthcare professional or healthcare provider that is responsible for one or more aspects of a patient's care; including, but not limited to, a doctor, a nurse, a physician's assistant, a pharmacist, a technician, and the like. The terms “provider” and “practitioner” may be used interchangeably throughout the present disclosure. As used herein, the term “practitioner user” refers to a provider/practitioner who is also a user of the inventive systems and methods described herein.

As used herein, the terms “progress note” or “clinical note” or “clinical progress note” refer to a medical record that details a patient's clinical status and/or achievements during the course of (e.g., at regular intervals) a treatment regimen or the provision of other medical care.

As used herein, the term “patient” refers to any recipient of health care services that are performed by, or facilitated by, a practitioner; including, but not limited to, an individual with at least one disease, disorder or other medical condition. As used herein, the term “patient user” refers to a patient who is also a user of the inventive systems and methods described herein.

As used herein, the terms “remote intervention” or “remote patient intervention” or refer to a model of care that incorporates the use of remote patient monitoring data via one or more networked devices to provide real-time disease and/or medication management for patients based on physician-approved protocols.

As used herein, the terms “remote patient interaction” or “digitally supported interaction,” or any combination thereof, refer to an interaction between a patient and one or more computing system interface (e.g., AI agent, application interface, etc.) in which the patient user provides one or more user-generated inputs (e.g., voice, text, etc.) in response to one or more prompts associated with, or related to, the provision of medical care to the patient.

As used herein, the term “clinical protocol” refers to a structured plan or set of guidelines that outlines the objectives, design, methodology, statistical considerations, and operational aspects of a clinical trial or medical intervention. An example of a clinical protocol may include, for example, a dosing regimen for at least one medication.

An exemplary system and method according to the principles herein may include an application configured to configure a clinical protocol (e.g., in accordance with one or more inputs from a practitioner user) for a remote patient intervention related to the treatment or management of one or more disease condition or disorder. The exemplary system and method of the present disclosure may further include a conversational AI engine configured to configure a conversational AI model (e.g., a large language model) according to one or more parameters of the clinical protocol. The exemplary system and method of the present disclosure may further include one or more data processing framework configured to receive conversational data generated via one or more multi-turn interactions (e.g., voice-based or text-based) between the patient user and a conversational AI agent. The one or more data processing framework may be further configured to ingest data from a plurality of clinical modalities, including, for example, electronic medical record (EMR) or electronic health record (EHR) data, wearable device data (e.g., continuous glucose monitor) and/or other user-generated data. The one or more data processing framework may be configured to format multiple independent data sources and/or data types (e.g., modalities) into a standardized format. The exemplary system and method of the present disclosure may further include a generative AI engine configured to generate a clinical note (e.g., a progress note) for the patient interaction based on the conversational data and the other data. The exemplary system and method of the present disclosure may further include one or more data transfer protocols for submitting the clinical note to one or more third-party server (e.g., a payor system server and/or an EMR/EMH server) in connection with submitting a medical billing claim for the patient interaction.

In accordance with an exemplary use case provided by embodiments of the present disclosure, a practitioner user may configure a clinical protocol for a remote patient intervention that is facilitated by a conversational AI agent. The conversational AI agent may execute the clinical protocol via a conversational interaction with the patient (e.g., via phone call, video call, live chat and the like). The conversational data (e.g., patient responses) may be processed to convert the conversational data into a standardized format. The conversational data, along with other medical record data for the patient, may be further processed via a generative AI engine to generate a clinical note for the patient based on the conversational interaction (e.g., according to the clinical protocol). The generative AI engine may be further configured to analyze the data to assign a medical billing code for the conversational interaction. The clinical note may be communicated to the practitioner user via an application interface. The practitioner user may electronically sign the clinical note via the application interface. The application server may submit the clinical note to a medical billing server or other third-party server to enable the practitioner user to generate a billing claim for the conversational interaction.

Certain benefits and advantages of the present disclosure include a method and system for automatically generating medical records for remote patient interventions.

Certain benefits and advantages of the present disclosure further include a method and system for automatically generating clinical notes for digitally supported interactions.

Certain benefits and advantages of the present disclosure further include a method and system for converting multiple types of medical data into a standardized format in order to automatically generate a medical record (e.g., a clinical note).

Certain benefits and advantages of the present disclosure further include a method and system for analyzing data generated from a remote patient interaction to assign a billing code for the remote patient interaction and submit a medical billing claim for the remote patient interaction.

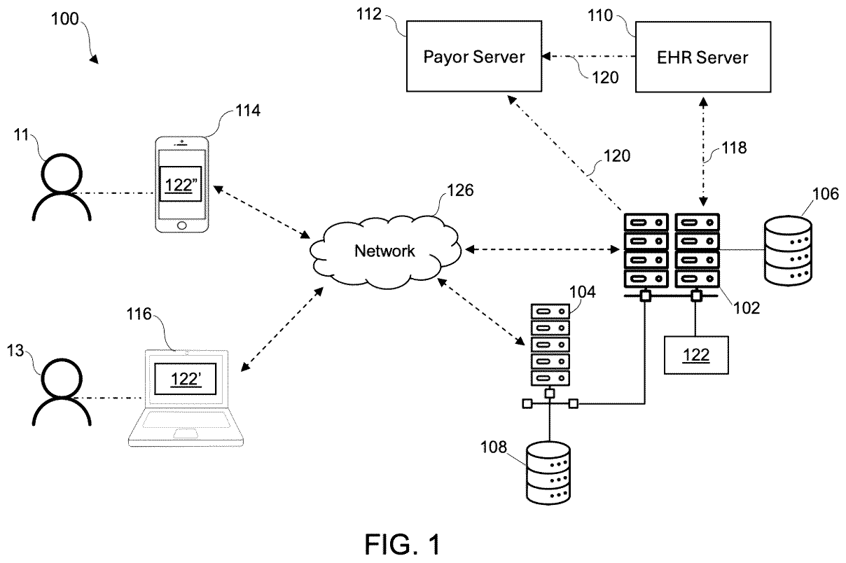

Turning now descriptively to the drawings, in which similar reference characters denote similar elements throughout the several views, FIG. 1 depicts an architecture diagram of a generative AI medical documentation system 100. In accordance with certain aspects of the present disclosure, system 100 is configured to facilitate the generation of certain medical records incident to a digitally supported interaction between a patient user and a conversational AI agent (e.g. a remote patient intervention). In accordance with certain aspects of the present disclosure, system 100 is configured to ingest and process data generated during a digitally supported interaction between a patient user and a conversational AI agent to automatically assign a billing code to the interaction based on the context of the interaction and autonomously generate a clinical note for the interaction. In accordance with certain aspects of the present disclosure, system 100 is configured to process data from one or more sources to automatically generate a clinical note for a digitally supported interaction between a patient user and a conversational AI agent.

In accordance with certain aspects of the present disclosure, system 100 comprises an application server 102 and a conversational AI server 104. In certain embodiments, conversational AI server 104 may be embodied as the same server (or group of servers) as application server 102 or may comprise a different server (or group of servers) as application server 102. In certain embodiments, conversational AI server 104 may comprise a third-party server (e.g., an external server associated with a different entity to that of application server 102). In certain embodiments, conversational AI server 104 and application server 102 may be communicably engaged via an application programming interface (API) to facilitate the transmission of data therebetween (e.g., in real-time). In certain embodiments, system 100 may further comprise an application database 106 communicably engaged with application server 102. System 100 may further comprise a conversational AI database 108 communicably engaged with conversational AI server 104. In accordance with certain aspects of the present disclosure, application server 102 may be configured to host a software application 122 comprising instructions for configuring a clinical protocol for a remote patient intervention for at least one patient and facilitating the remote patient intervention for the patient.

In accordance with certain aspects of the present disclosure, system 100 may further comprise a patient client 114 associated with a patient user 11. Patient client 114 may comprise a personal computing device, such as a smartphone, personal computer, tablet computer and the like. In certain embodiments, patient client 114 comprise a telephone configured to make voice calls via a telephony network. In certain embodiments, system 100 may comprise at least one peripheral device 124 communicably engaged with patient client 114 via a data transfer interface (e.g., BLUETOOTH low energy, near field communication, etc.). In certain embodiments, peripheral device 124 may comprise a device for collecting biometric data or activity data for patient user 11; for example, a wearable activity tracker, a continuous glucose monitor (CGM), a blood pressure cuff, a pulse monitor, and the like. System 100 may further comprise a practitioner client 116 associated with a practitioner user 13. In accordance with certain aspects of the present disclosure, practitioner user 13 may comprise a medical professional, and patient user 11 may comprise a patient whose medical care is supervised or facilitated by practitioner user 13. Practitioner client 116 may comprise a personal computing device, such as a computer workstation, smartphone, laptop, tablet computer, and the like. In accordance with certain aspects of the present disclosure, patient client 114 and practitioner client 116 may be communicably engaged with application server 102 and/or conversational AI server 104 via a network interface 126. Network interface 126 may comprise one or more network communication means, including, for example, Internet, cellular, telephony network, a local area network (LAN), a wide area network (WAN) and the like. In accordance with certain embodiments, application server 102 and/or conversational AI server 104 may be configured to execute telephone calls over network interface 126 (e.g., via a voice over Internet-protocol (VoIP) network (i.e., telephony network), cellular telephone network, and/or a public switched telephone network (PTSN)).

In accordance with certain aspects of the present disclosure, practitioner client 116 may be configured to execute a first instance 122′ of application 122. First instance 122′ of application 122 may comprise a user interface configured specifically for practitioner user 13. First instance 122′ of application 122 may comprise a plurality of graphical user interface elements configured to enable practitioner user 13 to configure at least one clinical protocol for patient user 11. In accordance with certain aspects of the present disclosure, the at least one clinical protocol may comprise a protocol for managing or treating at least one disease or medical condition. For example, the clinical protocol may include a medication dosing regimen for a disease such as Type-2 diabetes or hypertension. In accordance with certain aspects of the present disclosure, patient client 114 may be configured to execute a second instance 122″ of application 122. Second instance 122″ of application 122 may comprise a user interface configured specifically for patient user 11. Second instance 122″ of application 122 may comprise a plurality of graphical user interface elements configured to enable patient user 11 to view, manage and/or complete one or more tasks associated with the at least one clinical protocol. In accordance with certain embodiments, second instance 122″ is configured to enable a bi-directional interface between patient client 114 and conversational AI server 104. In certain embodiments, the bi-directional interface may comprise a chat interface, a voice call interface and/or a video call interface. In certain embodiments, patient user 11 may interface with conversational AI server 104 via a telephone call.

In accordance with certain aspects of the present disclosure, system 100 may further comprise an electronic health records (EHR) server 110. EHR server 110 may comprise a HIPAA-compliant server configured to store longitudinal medical records associated with patient user 11. In accordance with certain aspects of the present disclosure, EHR server 110 and application server 102 may be communicably engaged via API 118. API 118 may be configured to facilitate the transfer of documents, and other data, between EHR server 110 and application server 102. API 118 may be configured to facilitate a real-time data transfer interface between EHR server 110 and application server 102 and/or may be configured to facilitate one or more batch data transfer protocols between EHR server 110 and application server 102. In accordance with certain aspects of the present disclosure, system 100 may further comprise a payor server 112. In accordance with certain aspects of the present disclosure, payor server 112 may be associated with a medical payor entity; for example, a health insurance provider or Medicare/Medicaid. In accordance with certain embodiments, payor server 112 and application server 102 may be communicably engaged via an electronic data interchange (EDI) interface 120. EDI interface 120 may a secure internet protocol to enable the bi-directional sharing of documents between payor server 112 and application server 102. In certain embodiments, EDI interface 120 may comprise a standard document format by which application server 102 must format documents for transmission. In accordance with certain embodiments, EHR server 110 may also be communicably engaged with payor server 112 via EDI interface 120.

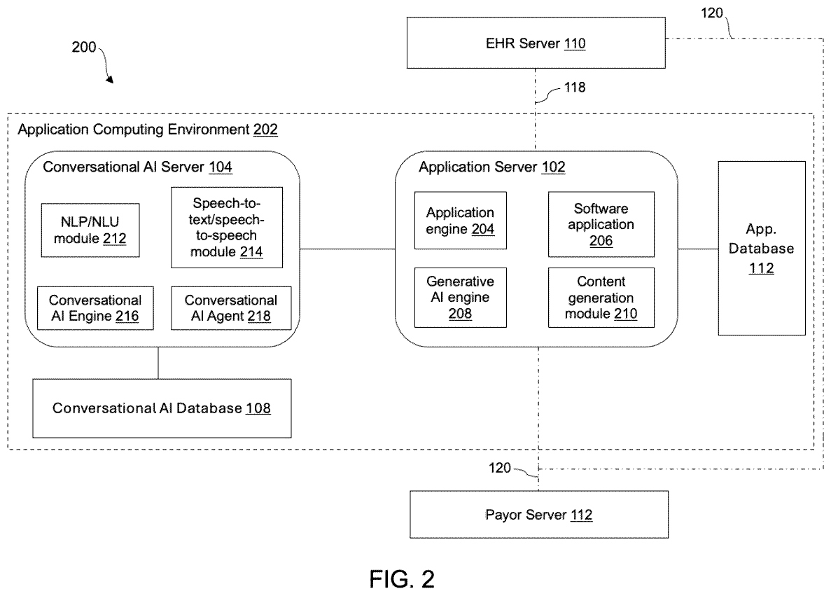

Referring now to FIG. 2, a system diagram of a generative AI medical documentation system 200 is shown. In accordance with certain aspects of the present disclosure, system 200 comprises an embodiment of system 100, as shown and described in association with FIG. 1. In accordance with certain aspects of the present disclosure, system 200 comprises an application computing environment 202 comprising application server 102, conversational AI server 104, application database 112 and conversational AI database 108. Application server 102 may comprise one or more computing module configured to facilitate one or more operations of system 200. In accordance with certain embodiments, the one or more computing modules may comprise an application engine 204, software application 122, a generative AI engine 208, and a content generation module 210. Application engine 204 may comprise a combination of hardware and software elements configured to execute and/or enable the operations of software application 122. Generative AI engine 208 may comprise a combination of hardware and software elements configured to execute and/or enable the operations of content generation module 210. Generative AI engine 208 may comprise a machine learning framework comprising a data processing model, or ensemble of models, and techniques that aim to generate new data or content that resembles human-created data. In certain embodiments, the machine learning framework may include, for example, one or more generative adversarial networks (GANs), variational autoencoders (VAEs), autoregressive models (e.g., large language models), recurrent neural networks, transformer-based models (e.g., generative pre-trained transformers (GPTs)), and reinforcement learning models. In certain embodiments, these models may work in combination with each other. For example, a reinforcement learning model may be implemented in conjunction with a transformer-based model to improve generative text through user feedback. Content generation module 210 may comprise a plurality of encoded operations for content generation according to one or more standardized formats and/or content parameters.

In accordance with certain aspects of the present disclosure, content generation module 210 is configured to generate a clinical note according to a plurality of parameters associated with a clinical protocol. Content generation module 210 may be configured to generate the clinical note in accordance with one or more medical billing parameters. In accordance with certain aspects of the present disclosure, content generation module 210 is operably engaged with generative AI engine 208 to generate the clinical note. In accordance with certain embodiments, generative AI engine 208 is configured to generate content for the clinical note (e.g., a summary of a patient interaction and/or a patient history summary) based on data collected through one or more synchronous or asynchronous patient interaction and/or one or more stored medical records for the patient. Content generation module 210 may be configured to receive and process a plurality of generative text from generative AI engine 208 to generate the clinical note (e.g., according to one or more standardized format).

In accordance with certain aspects of the present disclosure, system 200 may further comprise EHR server 110 and payor server 112. EHR server 110 may be communicably engaged with application server 102 via API 118. In accordance with certain embodiments, API 118 enables a real-time data transfer interface between EHR server 110 and application server 102 to enable the transfer of medical records data therebetween. In certain embodiments, EHR server 110 may comprise a HIPAA-compliant server. Examples of EHR server 110 may include EHR systems provided by companies such as EPIC SYSTEMS (Verona, WI) and ORACLE HEALTH (Kansas City, MO). In certain embodiments, payor server 112 may be communicably engaged with application server 102 via EDI interface 120. In accordance with certain embodiments, content generation module 210 may be configured to format the clinical note according to one or more document formatting parameters defined by EDI interface 120. In certain embodiments, EHR server 110 may be communicably engaged with payor server 112 via EDI interface 120. In accordance with certain aspects of the present disclosure, the clinical note comprises a medical billing claim for at least one synchronous or asynchronous interaction between a patient user (e.g., patient user 11, as shown and described in FIG. 1) and a conversational AI agent 218.

In accordance with certain aspects of the present disclosure, at least one synchronous or asynchronous interaction between the patient user and conversational AI agent 218 is facilitated via conversational AI server 104. In accordance with certain embodiments, conversational AI server 104 comprises a natural language processing (NLP) and/or natural language understanding (NLP/NLU) module 212, a speech-to-text and/or speech-to-speech (STT/STS) module 214, a conversational AI engine 216, and conversational AI agent 218. In accordance with certain embodiments, NLP/NLU module 212 may comprise a machine learning framework configured to process natural language data (e.g., via a patient interaction with conversational AI agent 218) and interpret natural language data to derive meaning, identify context, and draw insights therefrom. In accordance with certain embodiments, NLP/NLU module 212 may comprise one or more NLP algorithms configured to process free form natural language text and transform it into a standardized format for use by one or more other computing modules residing on conversational AI server 104. This may include one or more pre-processing steps or operations including, for example, parsing, stop-word removal, part-of-speech (POS) tagging, tokenization, and the like. NLP/NLU module 212 may comprise one or more NLU algorithms configured to process the natural language input to perform functions including, for example, sentiment identification, Name Entity Recognition, semantic processing, and the like. In accordance with certain embodiments, the one or more NLU algorithms operate on text that has already been standardized by the one or more NLP algorithms.

In accordance with certain embodiments, STT/STS module 214 may comprise one or more algorithms configured to convert data from a text format to a machine voice format. STT/STS module 214 may be operably engaged with conversational AI engine 216 and conversational AI agent 218 to enable conversational AI agent 218 to output a plurality of machine-voice prompts to a patient user via a bi-directional voice interface (e.g., a phone call or video call). STT/STS module 214 may comprise a speech-to-speech engine comprising an AI framework including one or more algorithms and deep learning techniques configured to recognize and interpret voice utterances from the patient user and enable machine voice responses by conversational AI agent 218. The speech-to-speech engine may be configured to reduce latency in real-time interactions between the patient user and conversational AI agent 218. In accordance with certain aspects of the present disclosure, conversational AI agent 218 may be operably engaged with conversational AI engine 216 to generate a plurality of voice-based prompts to enable one or more multi-turn conversational interactions between the patient user and conversational AI agent 218. In accordance with certain aspects of the present disclosure, conversational AI engine 216 is operably engaged with NLP/NLU module 212 to analyze a user-generated input from the patient user to identify key words, context, and intent via one or more data processing operations, such as tokenization, parsing, and sentiment analysis. Conversational AI engine 216 may comprise one or more pre-trained models and contextual algorithms to process the user-generated input and determine/generate a natural language response. In accordance with certain embodiments, the one or more pre-trained models and contextual algorithms are configured according to one or more parameters defined by a clinical protocol. Once a response is generated, conversational AI agent 216 may present the response to the patient user via a bi-directional conversational interface (e.g., a text chat, a voice call, and/or a video call). Conversational prompts/responses and user-generated conversational data may be stored in conversational AI database. Conversational AI engine 216 may incorporate historical conversational data into its training data to improve its interactions with the patient user over time (e.g., to make the conversational interaction more personalized and accurate for the patient user). In accordance with certain aspects of the present disclosure, conversational AI engine 216 may be configured to process the conversational data from an interaction between conversational AI agent 218 and the patient to generate a transcript of the interaction. Transcript data may be communicated between conversational AI server 104 and application server 102. In accordance with certain aspects of the present disclosure, application server 102 may be configured to process the transcript data to generate the clinical note (e.g., in accordance with the above-described operations).

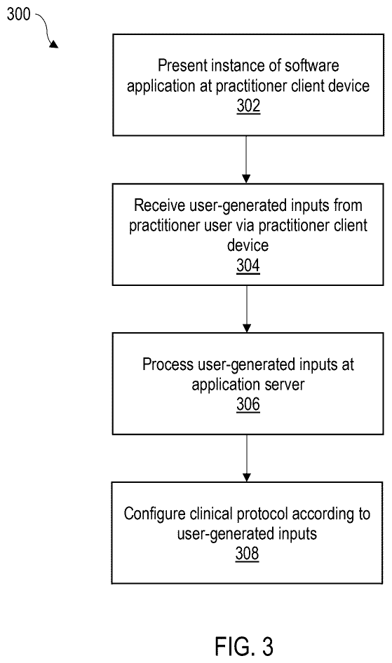

Referring now to FIG. 3, a process flow diagram of a routine 300 of a generative AI medical documentation system is shown. In accordance with certain aspects of the present disclosure, the generative AI medical documentation system may comprise system 100, as shown and described in association with FIG. 1, and/or the generative AI medical documentation system may comprise system 200, as shown and described in association with FIG. 2. Accordingly, routine 300 may be embodied within one or more system operations of system 100 (FIG. 1) or system 200 (FIG. 2). The operations in routine 300 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 300 comprises one or more steps or operations 302-308 for configuring a clinical protocol according to a plurality of user-generated inputs from a practitioner user (e.g., practitioner user 11 as shown and described in association with FIG. 1). In accordance with certain embodiments, routine 300 may comprise one or more steps or operations for presenting an instance of a software application at a practitioner client device (Step 302). The instance of the software application may include practitioner instance 122′ of FIG. 1. In accordance with certain embodiments the instance of the software application may comprise a graphical user interface configured to enable the practitioner user to configure the clinical protocol for a specific patient (e.g., patient user 11 of FIG. 1). In accordance with certain aspects of the present disclosure, the clinical protocol is associated with at least one intervention designed to treat, prevent or manage at least one medical condition of the patient user. For example, the clinical protocol may comprise a medication dosing regimen for management of Type-2 diabetes for the patient user. The clinical protocol may include, for example, one or more conditions upon which the patient user may initiate, titrate and/or terminate one or more diabetes medication (for example, a glucagon-like peptide-1 (GLP-1) drug, a biguanide drug, sodium glucose cotransporter 2 (SGLT2) drug, and the like). Routine 300 may proceed by performing one or more steps or operations for receiving the user-generated inputs from the practitioner user at the practitioner client device (e.g., via the graphical user interface) (Step 304). Routine 300 may proceed further by performing one or more steps or operations for processing the user-generated inputs at an application server (e.g., application server 102 as shown and described in FIGS. 1 and 2) according to one or more data processing operations (Step 306). Routine 300 may proceed further by performing one or more steps or operations for configuring the clinical protocol according to the user-generated inputs (Step 308). The steps and/or parameters of the clinical protocol may be stored in an application database communicably engaged with the application server (e.g., application database 108 as shown and described in FIGS. 1 and 2).

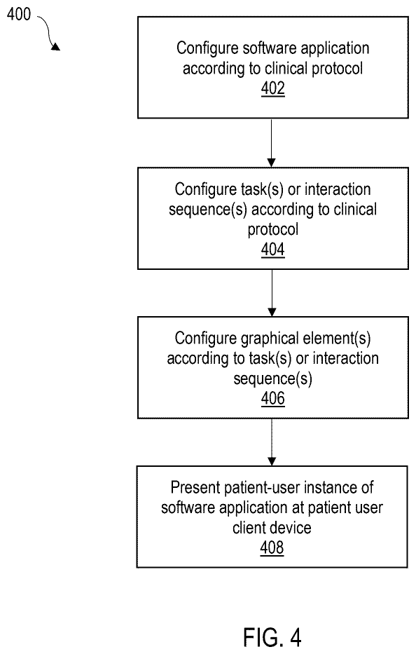

Referring now to FIG. 4, a process flow diagram of a routine 400 of a generative AI medical documentation system is shown. In accordance with certain aspects of the present disclosure, the generative AI medical documentation system may comprise system 100, as shown and described in association with FIG. 1, and/or the generative AI medical documentation system may comprise system 200, as shown and described in association with FIG. 2. Accordingly, routine 400 may be embodied within one or more system operations of system 100 (FIG. 1) or system 200 (FIG. 2). In accordance with certain embodiments, the operations in routine 400 may be successive or sequential to one or more operations of routine 300 (FIG. 3). The operations in routine 400 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 400 may comprise one or more steps or operations 402-408 for configuring a software application according to a clinical protocol (e.g., the clinical protocol configured in accordance with routine 300 of FIG. 3) and presenting a patient-user instance of the software application (e.g., second instance 122″ of FIG. 1) to a patient user via a patient client device (e.g., patient client 114 of FIG. 1). In accordance with certain aspects of the present disclosure, routine 400 may comprise one or more steps or operations for configuring one or more routines or user workflows of a software application according to the clinical protocol (Step 402). In accordance with certain aspects of the present disclosure, the software application comprises an application for managing and implementing a remote patient intervention for the patient user. The remote patient intervention may include, for example, one or more steps or operations for implementing the clinical protocol via one or more networked systems and devices. Routine 400 may proceed by executing one or more steps or operations for configuring one or more tasks or interaction sequences within the software application for the end user according to the clinical protocol (Step 404). Step 404 may comprise one or more steps or operations for configuring the one or more tasks or interaction sequences according to one or more states of the clinical protocol. For example, the one or more task may comprise required tasks, such as inputting patient biological data (e.g., HbA1c data) and medication log data (e.g., timing and dosage of medication). The one or more interaction sequences may include, for example, one or more scheduled interaction between the conversational AI agent and the patient user (e.g., to prompt the user for specific data points). In accordance with certain embodiments, routine 400 may proceed by executing one or more steps or operations for configuring one or more graphical elements for the patient-user instance of the software application (Step 406). Routine 400 may proceed by executing one or more steps or operations for presenting the patient-user instance of the software application to the patient user at the patient user client device (Step 408). In accordance with certain embodiments, the patient-user instance may be configured to present one or more prompts, graphical elements, or the like configured to facilitate the one or more tasks or interaction sequences between the conversational AI agent and the patient user.

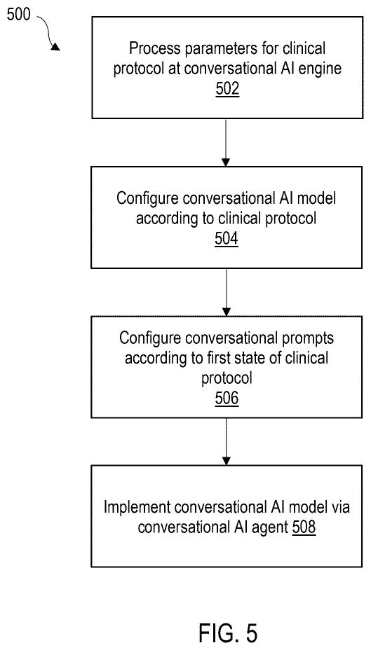

Referring now to FIG. 5, a process flow diagram of a routine 500 of a generative AI medical documentation system is shown. In accordance with certain aspects of the present disclosure, the generative AI medical documentation system may comprise system 100, as shown and described in association with FIG. 1, and/or the generative AI medical documentation system may comprise system 200, as shown and described in association with FIG. 2. Accordingly, routine 500 may be embodied within one or more system operations of system 100 (FIG. 1) or system 200 (FIG. 2). In accordance with certain embodiments, the operations in routine 500 may be successive or sequential to one or more operations of routine 300 (FIG. 3) or routine 400 (FIG. 4). The operations in routine 500 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 500 may comprise one or more steps or operations 502-508 for configuring a conversational AI model according to a clinical protocol (e.g., the clinical protocol configured in accordance with routine 300 of FIG. 3) and implementing the conversational AI model at a conversational AI agent (e.g., conversational AI agent 218 of FIG. 2). In accordance with certain aspects of the present disclosure, routine 500 may comprise one or more steps or operations for processing one or more parameters for the clinical protocol via a conversational AI engine (e.g., conversational AI engine 216 of FIG. 2) (Step 502). The one or more parameters may comprise one or more objectives, design aspects, methodologies, statistical considerations, and/or operational aspects of the clinical protocol. Step 502 may comprise one or more steps or operations for analyzing the one or more parameters to extract one or more contextual aspects of the clinical protocol according to one or more pre-trained models and contextual algorithms. Routine 500 may proceed by executing one or more steps or operations for configuring the conversational AI model according to the parameters of the clinical protocol (Step 504). Step 504 may include one or more steps or operations for configuring one or more dependent variables in the conversational AI model according to the one or more parameters of the clinical protocol. Routine 500 may proceed by executing one or more steps or operations for configuring one or more conversational prompts according to a first state of the clinical protocol (Step 506). In accordance with certain embodiments, the one or more conversational prompts may comprise one or more prompts configured to elicit specific information from the patient user via one or more interaction with the patient user. For example, the one or more prompts may be configured (e.g., according to the clinical protocol) to elicit information such as blood glucose data, heart rate data, blood pressure data, medication log data, and the like. In accordance with certain embodiments, the first state of the clinical protocol may be associated with a discrete phase of the clinical protocol or a first task, or group of tasks, in a sequential series of tasks. For example, a first state of the clinical protocol may comprise a medication dosing regimen that is applicable under a first set of conditions (e.g., fasting blood glucose levels for the patient, current medication dosage, and the like). In accordance with certain aspects of the present disclosure, if the first set of conditions change or are no longer applicable, the system may be configured to transition the clinical protocol from the first state to a second or subsequent state. In response to one or more steps or operations in Step 506, routine 500 may proceed by executing one or more steps or operations for implementing the conversational AI model via the conversational AI agent (Step 508). Step 508 may comprise one or more steps or operations for executing one or more multi-turn conversational interactions between the conversational AI agent and the patient user. In certain embodiments, the one or more multi-turn conversational interactions may be implemented via a bi-directional voice call. The bi-directional voice call may be executed via a telephone call (e.g. VoIP) between the conversational AI agent and the patient client device (e.g., telephone). In certain embodiments, the bi-directional voice call may comprise a video call that is implemented via an interface of the patient instance of the software application. In certain embodiments, the one or more multi-turn conversational interactions may be implemented via a chat interface presented at the patient instance of the software application.

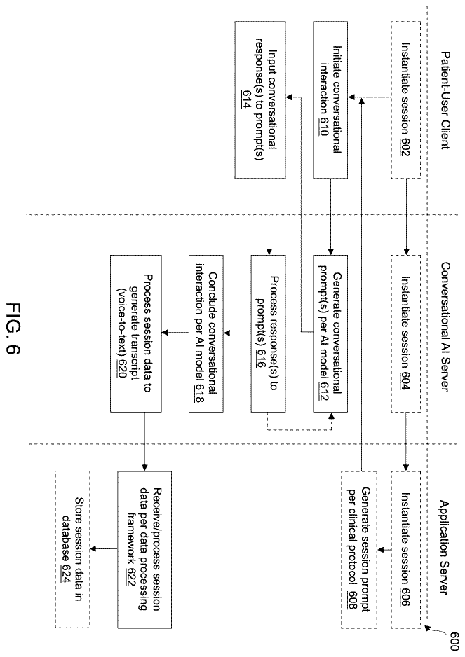

Referring now to FIG. 6, a process flow diagram of a routine 600 of a generative AI medical documentation system is shown. In accordance with certain aspects of the present disclosure, the generative AI medical documentation system may comprise system 100, as shown and described in association with FIG. 1, and/or the generative AI medical documentation system may comprise system 200, as shown and described in association with FIG. 2. Accordingly, routine 600 may be embodied within one or more system operations of system 100 (FIG. 1) or system 200 (FIG. 2). In accordance with certain embodiments, the operations in routine 600 may be successive or sequential to one or more operations of routine 300 (FIG. 3) or routine 400 (FIG. 4) or routine 500 (FIG. 5). The operations in routine 600 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 600 comprises one or more steps or operations 602-624 for instantiating a conversational session between a patient user and a conversational AI agent and generating a text transcript of the multi-turn conversational interactions between the patient user and the conversational AI agent. In certain embodiments, the conversational session may be instantiated via the patient user instance (e.g., patient user instance 122″ of FIG. 1) of the software application (e.g., software application 122 of FIG. 1). In certain embodiments, the conversational session may be instantiated via a telephone call (e.g., outside of an instance of the software application) between a telephone associated with the end user and the conversational AI agent (e.g., via a voice-over-IP network interface). In accordance with certain aspects of the present disclosure, routine 600 may begin by executing one or more steps or operations for instantiating a session of a multi-turn conversational interaction at a patient-user client (Step 602) and/or a conversational AI server (Step 604) and/or an application server (Step 606). In certain embodiments, the conversational AI server and the application server may comprise the same server. In certain embodiments, a session is instantiated at the patient user client in response to the patient user making a phone call to a designated telephone number or receiving a phone call from the conversational AI server (e.g., via the conversational AI agent). In accordance with certain embodiments and/or instances, routine 600 may comprise one or more steps or operations for generating a session prompt according to the clinical protocol at the application server (e.g., via the application software engine) (Step 608). The session prompt may comprise a prompt configured to invoke the patient user to initiate a session with the conversational AI agent. For example, the session prompt may comprise a push notification at the patient user's client device comprising a message such as, “It's time to check-in-would you like to begin your check-in session now?” Alternatively, the session prompt may comprise a telephone call in which the conversational AI agent provides a voice prompt such as, “It's time to check-in-if would you like to begin your check-in session now, say ‘YES’.”

In accordance with certain aspects of the present disclosure, routine 600 may proceed by executing one or more steps or operations for initiating a bi-directional conversational interaction between the patient user (e.g., at the patient-user client device) and the conversational AI agent (Step 610). In certain embodiments, the patient user may initiate the conversation by providing an invocation word or phrase or providing an affirmative response to a prompt provided by the conversational AI agent or a push notification. Routine 600 may proceed by executing one or more steps or operations for generating (e.g., via the conversational AI agent) a conversational prompt according to the conversational AI model and providing the prompt to the patient user via the patient user client device (Step 612). In accordance with certain aspects of the present disclosure, the conversational prompt is configured according to the clinical protocol. In certain embodiments, the conversational prompt comprises a machine voice output via a bi-directional voice call between the conversational AI agent and the patient user. Routine 600 may proceed by executing one or more steps or operations for receiving one or more conversational responses from the patient user (e.g., at the patient-user client device) in response to the conversational prompt(s) (e.g., from Step 612) (Step 614). In certain embodiments, the one or more conversational responses from the patient user may comprise one or more voice inputs. In certain embodiments, the one or more conversational inputs from the patient user may comprise one or more text inputs. Routine 600 may proceed by executing one or more steps or operations for processing the one or more conversational responses from the patient user according to the conversational AI model (Step 616). In accordance with certain aspects of the present disclosure, Step 616 comprises one or more steps or operations for interpreting the one or more conversational responses and crafting one or more response according to the conversational AI model. If the conversational AI model determines that additional follow-up prompts are warranted (e.g., in accordance with the clinical protocol) then routine 600 will proceed to Step 612 to continue the multi-turn interaction between the patient and the conversational AI agent. If the conversational AI model determines that all necessary data inputs have been received from the patient user pursuant to the conversational interaction, then routine 600 may proceed by executing one or more steps or operations for concluding the conversational interaction in accordance with the conversational AI model (Step 618). For example, Step 618 may comprise presenting a concluding prompt to the patient user, such as, “Thank you for providing your responses, I will follow-up with your again in two days; good-bye.”

In accordance with certain aspects of the present disclosure, routine 600 may proceed by executing one or more steps or operations for processing the session data from the phone call or chat interface to generate a transcript of the conversational interaction between the patient user and the conversational AI agent (Step 620). Routine 600 may proceed by executing one or more steps or operations for receiving the session data (e.g. conversational transcript) at the application server and processing the session data according to at least one data processing framework (Step 622). In accordance with certain aspects of the present disclosure, the at least one data processing framework is configured according to the clinical protocol. In accordance with certain aspects of the present disclosure, routine 600 may conclude by executing one or more steps or operations for storing the session data in an application database (e.g., application database 106 of FIG. 1) (Step 624).

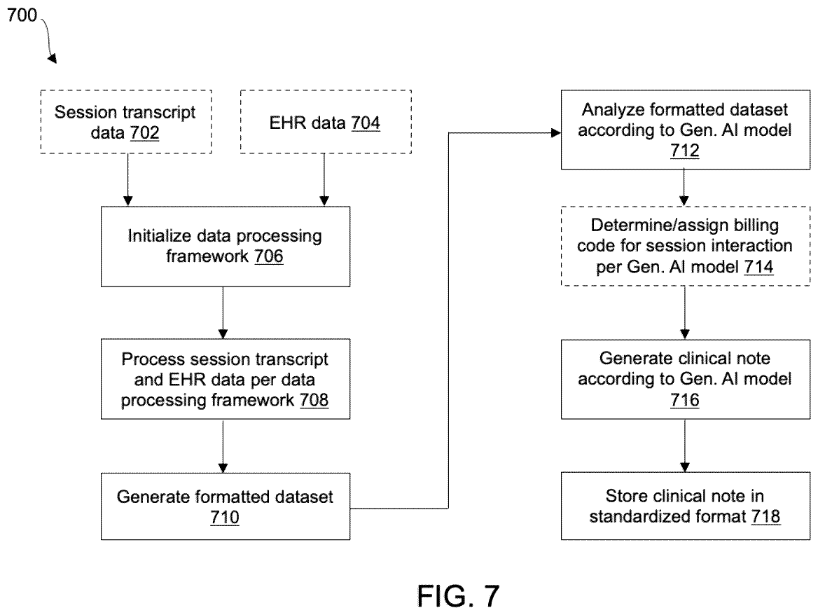

Referring now to FIG. 7, a process flow diagram of a routine 700 of a generative AI medical documentation system is shown. In accordance with certain aspects of the present disclosure, the generative AI medical documentation system may comprise system 100, as shown and described in association with FIG. 1, and/or the generative AI medical documentation system may comprise system 200, as shown and described in association with FIG. 2. Accordingly, routine 700 may be embodied within one or more system operations of system 100 (FIG. 1) or system 200 (FIG. 2). In accordance with certain embodiments, the operations in routine 700 may be successive or sequential to one or more operations of routine 300 (FIG. 3) or routine 400 (FIG. 4) or routine 500 (FIG. 5) or routine 600 (FIG. 6). The operations in routine 700 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 700 comprises one or more steps or operations 702-718 for formatting session transcript data and EHR data for a patient user to generate a clinical note for a remote patient intervention between a conversational AI agent and a patient user. Routine 700 may be initialized by executing one or more steps or operations for initializing a data processing framework (Step 706) to receive and process session transcript data 702 and EHR data 704. Step 706 may comprise one or more data pre-processing steps (e.g., data cleaning or data formatting). In accordance with certain aspects of the present disclosure, session transcript data 702 may comprise a transcript of a conversational interaction between the patient user and the conversational AI agent generated in accordance with routine 600 of FIG. 6. In accordance with certain embodiments, EHR data 704 comprises electronic health record data (e.g., electronic medical record data) for the patient user. EHR data 704 may comprise data collected from one or more prior interactions between the patient user and the conversational AI agent. EHR data 704 may further comprise one or more longitudinal health record data and/or laboratory testing data for the patient user. In accordance with certain aspects of the present disclosure, routine 700 may proceed by executing one or more steps or operations for processing the session transcript data (Step 708). In accordance with certain aspects of the present disclosure, Step 708 may comprise one or more operations to generate a formatted dataset (Step 710). In accordance with certain embodiments, the one or more operations may include, but are not limited to, operations for data cleaning (e.g., removing noise, handling duplicate data, and dealing with missing values); data transformation (e.g., text normalization and standardization, tokenization, and feature scaling); vectorization (e.g., transforming the data into vector forms suitable for input into the generative AI model); and feature extraction (e.g., extracting features or patterns from the raw data to reduce dimensionality).

In accordance with certain aspects of the present disclosure, routine 700 may proceed by executing one or more steps or operations for analyzing the formatted dataset (e.g., according to the output of Step 710) according to a generative AI model (e.g., via generative AI engine 208 of FIG. 2) (Step 712). In accordance with certain aspects of the present disclosure, the generative AI model comprises one or more machine learning algorithms configured to process the formatted dataset to generate an AI-created clinical note for the patient's interaction with the conversational AI agent (i.e., remote patient intervention). In certain embodiments, routine 700 comprises one or more steps or operations for determining (e.g., according to the generative AI model) and assigning a billing code for the patient's interaction with the conversational AI agent (Step 714). In accordance with certain aspects of the present disclosure, the generative AI model may be configured to extract and analyze one or more features from the formatted dataset to determine the appropriate billing code (or codes) for the remote patient intervention and assign the billing code(s) to the formatted dataset. In certain embodiments, Step 714 may be omitted (e.g., for embodiments in which the billing codes are already known). In accordance with certain aspects of the present disclosure, routine 700 may comprise one or more steps or operations for generating a clinical note from the formatted dataset according to the generative AI model (Step 716). In accordance with certain aspects of the present disclosure, the clinical note may include the assigned billing code(s). In accordance with certain aspects of the present disclosure, the generative AI model is configured to generate the clinical note to document a plurality of variables for the remote patient intervention. In certain embodiments, the generative AI model is configured to generate the clinical note to include documentation of medical necessity for the remote patient intervention including, for example, a summary of the patient's condition, symptoms, current stage of the clinical protocol, present diagnosis, and the care provided during the remote patient intervention. In certain embodiments, the generative AI model is configured to generate the clinical note to include a level of service determination for the remote patient intervention including, for example, the extent of the history with the patient, the complexity of medical decision-making and time spent with the patient. In certain embodiments, the generative AI model is configured to generate the clinical note to include chronic care documentation including, for example, documentation of blood sugar levels, treatment plans, and medication adjustments. In accordance with certain aspects of the present disclosure, routine 700 may comprise one or more steps or operations for storing the clinical note (e.g., in a database) according to at least one standardized document format (Step 718). In certain embodiments, the standardized document format may comprise an electronic data interchange format.

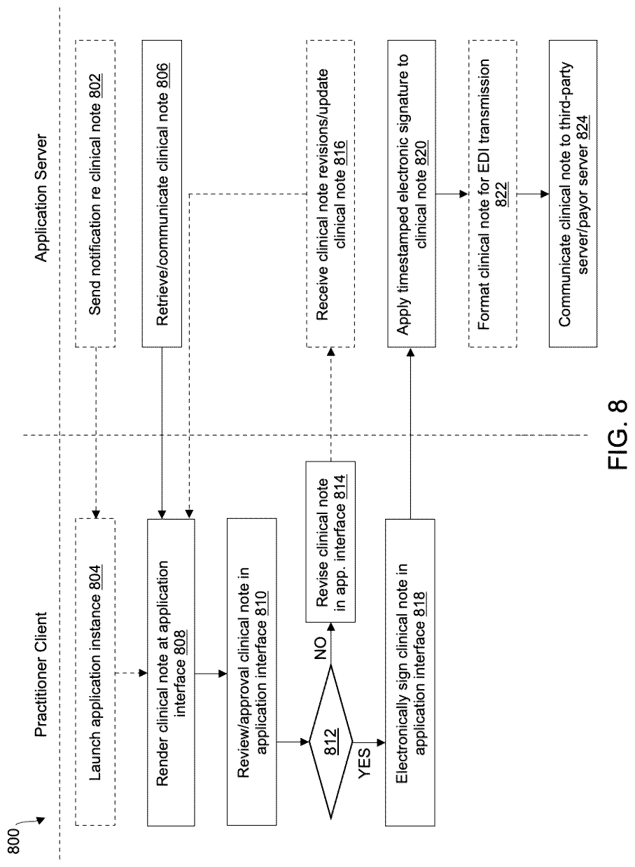

Referring now to FIG. 8, a process flow diagram of a routine 800 of a generative AI medical documentation system is shown. In accordance with certain aspects of the present disclosure, the generative AI medical documentation system may comprise system 100, as shown and described in association with FIG. 1, and/or the generative AI medical documentation system may comprise system 200, as shown and described in association with FIG. 2. Accordingly, routine 800 may be embodied within one or more system operations of system 100 (FIG. 1) or system 200 (FIG. 2). In accordance with certain embodiments, the operations in routine 800 may be successive or sequential to one or more operations of routine 300 (FIG. 3) or routine 400 (FIG. 4) or routine 500 (FIG. 5) or routine 600 (FIG. 6) or routine 700 (FIG. 7). The operations in routine 800 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 800 comprises one or more steps or operations 802-824 for communicating a clinical note to the provider user and communicating the clinical note to at least one third-party server. In accordance with certain aspects of the present disclosure, routine 800 may be initiated upon executing one or more steps or operations for sending a communication regarding the creation of a clinical note from the application server to the practitioner client (Step 802). The notification may comprise a push notification, an email notification, an in-app notification, an automated phone call, or another type of notification facilitated via a networked computing environment. Routine 800 may comprise one or more steps or operations for launching an instance of a software application (e.g., software application 122) at a practitioner user client device (Step 804). Routine 800 may comprise one or more steps or operations for retrieving/communicating the clinical note (e.g., in response to an input by the practitioner user at the application interface) (Step 806) and rendering the clinical note at a graphical user interface of the instance of the software application (Step 808). Routine 800 may comprise one or more steps or operations for the practitioner user to review the clinical note (e.g., via the graphical user interface) and determine whether to approve or revise the clinical note (Step 810). Routine 800 may comprise a decision step 812 based on whether the practitioner user approves the clinical note. If NO (i.e., the practitioner user does not approve the clinical note), routine 800 may proceed by executing one or more steps or operations to enable the practitioner user to revise the clinical note in the application interface (Step 814). Routine 800 may comprise one or more (optional) steps or operations for receiving the revisions to the clinical note at the application server and updating the clinical note at the application (Step 816). In said embodiments, routine 800 may proceed by rendering the revised clinical note at the application interface in accordance with Step 808). If YES (i.e., the practitioner user approves the clinical note), routine 800 may proceed by executing one or more steps or operations to enable the practitioner user to electronically sign the clinical note in the application interface (Step 818). In accordance with certain embodiments, routine 800 comprises one or more steps or operations for receiving the electronic signature at the application server and applying a timestamped electronic signature by the practitioner user to the clinical note at the application server (Step 820). In certain embodiments, Step 820 includes one or more steps or operations to hash the clinical note after the electronic signature is applied (i.e., convert the clinical note to a fixed-size string) and/or encrypt the clinical note to ensure the clinical note document file and the electronic signature remain secure. In certain embodiments, Step 820 includes one or more steps or operations for recording metadata for the electronic signature including, for example, a timestamp, geolocation, IP address, practitioner user interaction data, and the like. Step 820 may further include one or more steps or operations for embedding a visual indicator to the clinical note to indicate that it has been approved/signed by the practitioner user. In certain embodiments, routine 800 may comprise one or more steps or operations for formatting the clinical note for transmission via an EDI interface (Step 822). In certain embodiments, routine 800 may comprise one or more steps or operations for communicating the clinical note to a third-party server and/or a payor party server (Step 824). In certain embodiments, the third-party server may include the EHR server, and the payor party server may include an insurance company server or a government health service server.

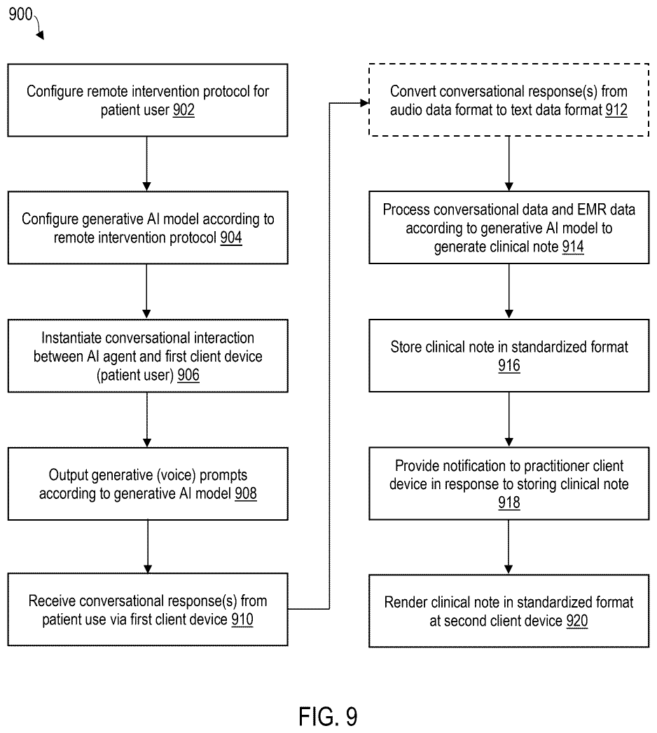

Referring now to FIG. 9, a process flow diagram of a generative AI medical billing method 900 is shown. In accordance with certain aspects of the present disclosure, method 900 may be embodied within one or more operations or routines of system 100, as shown and described in association with FIG. 1, and/or system 200, as shown and described in association with FIG. 2. In accordance with certain embodiments, method 900 may be embodied as one or more steps or operations of routine 300 (FIG. 3), routine 400 (FIG. 4), routine 500 (FIG. 5), routine 600 (FIG. 6), routine 700 (FIG. 7) and/or routine 800 (FIG. 8). The steps in method 900 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, method 900 may comprise one or more steps 902-920 for automatically formatting medical data for remote patient interventions. In accordance with certain embodiments, method 900 may comprise one or more steps or operations for configuring (e.g., with at least one server) a remote intervention protocol for a patient (Step 902). Method 900 may further comprise one or more steps or operations for configuring (e.g., with the at least one server) a generative artificial intelligence (AI) model according to the remote intervention protocol for the patient (Step 904). In accordance with certain embodiments, method 900 may further comprise one or more steps or operations for instantiating (e.g., with the at least one server) a conversational interaction between a conversational AI agent and a first client device associated with the patient (e.g., wherein the first client device comprises an audio input/output means) (Step 906). In accordance with certain embodiments, method 900 may further comprise one or more steps or operations for outputting (e.g., with the conversational AI agent) one or more generative prompts according to the generative AI model at the first client device (Step 908). In accordance with certain embodiments, method 900 may further comprise one or more steps or operations for receiving (e.g., with the at least one server) one or more conversational responses from the patient via the first client device (Step 910). In accordance with certain embodiments, method 900 may (optionally) comprise one or more steps or operations for converting (e.g., with the at least one server) the one or more conversational responses from an audio data format to a text data format (e.g., wherein the text data format comprises a transcript of the conversational interaction between the conversational AI agent and the patient) (Step 912). In accordance with certain embodiments, method 900 may further comprise one or more steps or operations for processing (e.g., with the at least one server) the transcript of the conversational interaction and one or more electronic medical records for the patient according to the generative AI model to generate a clinical note for the patient (Step 914). In accordance with certain embodiments, method 900 may further comprise one or more steps or operations for storing (e.g., in a non-transitory storage device communicably engaged with the at least one server) the clinical note for the patient in a standardized format (Step 916). In accordance with certain embodiments, method 900 may further comprise one or more steps or operations for providing (e.g., with the at least one server) a notification to a second client device associated with a provider user in response to storing the clinical note (Step 918). In accordance with certain embodiments, method 900 may further comprise one or more steps or operations for rendering (e.g., with the at least one server) the clinical note in the standardized format at the second client device (Step 920).

In accordance with certain embodiments, method 900 may further comprise one or more steps or operations for receiving (e.g., at a user interface of the second client device) an electronic signature for the provider user in response to rendering the clinical note. In certain embodiments, method 900 may further comprise one or more steps or operations for applying (e.g., with the at least one server) the electronic signature for the provider user to the clinical note and storing the signed clinical note in the standardized format. In certain embodiments, the standardized format may comprise an electronic data interchange format for at least one electronic medical billing system. In certain embodiments, method 900 may further comprise one or more steps or operations for communicating (e.g., with the at least one server) the signed clinical note to at least one third-party server via an electronic data interchange. In certain embodiments, method 900 may further comprise one or more steps or operations for initiating (e.g., with the at least one server) at least one routine of the remote intervention protocol in response to at least one user-generated input by the patient at the first client device. In certain embodiments, method 900 may further comprise one or more steps or operations for processing (e.g., with the at least one server) the transcript of the conversational interaction and the one or more electronic medical records for the patient to determine one or more medical billing code for the conversational interaction. In certain embodiments, method 900 may further comprise one or more steps or operations for assigning (e.g., with the at least one server) the one or more determined medical billing code for the conversational interaction to the clinical note according to the standardized format. In certain embodiments, method 900 may further comprise one or more steps or operations for receiving (e.g., via the second client device) one or more user-generated inputs from the provider user (e.g., wherein the one or more user-generated inputs comprise one or more additions or revisions to the clinical note). In certain embodiments, method 900 may further comprise one or more steps or operations for updating (e.g., with the at least one server) the clinical note according to the one or more user-generated inputs from the provider user and storing the updated clinical note in the non-transitory storage device.

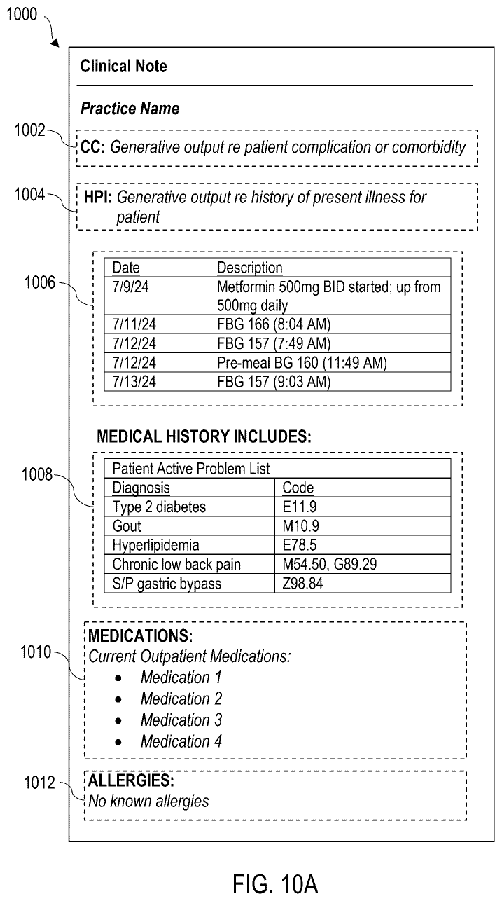

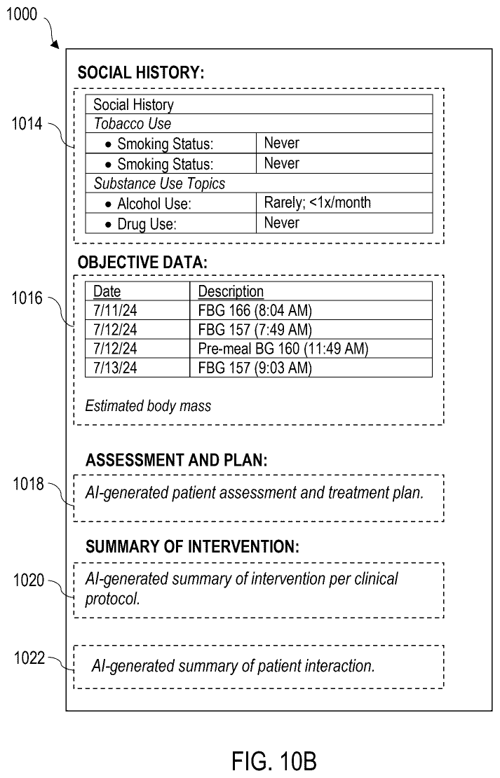

FIGS. 10A-10B are illustrations of an AI-generated clinical note 1000. In accordance with certain aspects of the present disclosure, clinical note 1000 may be generated as an output of system 100 (as shown and described in FIG. 1) and system 200 (as shown and described in FIG. 2). In accordance with certain aspects of the present disclosure, clinical note 1000 may be generated in accordance with one or more steps or operations of routine 300 (FIG. 3), routine 400 (FIG. 4), routine 500 (FIG. 5), routine 600 (FIG. 6), routine 700 (FIG. 7) and/or routine 800 (FIG. 8).

As shown in FIGS. 10A-10B, clinical note 1000 may comprise a plurality of generative text sections 1002-1022 comprising documentation of a remote patient intervention between a patient user and a conversational AI agent. The plurality of generative text sections 1002-1022 may comprise one or more patient history generated from longitudinal medical record data and/or data from one or more prior interactions between the patient user and the conversational AI agent. In accordance with certain embodiments, clinical note 1000 may comprise a first generative text section 1002 comprising a generative text output including data related to complications or comorbidities for the patient user. This data may be derived from the patient user's electronic medical record. In certain embodiments, clinical note 1000 may comprise a second generative text section 1004 comprising generative text output including data related to a history of present illness for the patient. This data may be derived from the patient user's electronic medical record and/or one or more prior interactions between the patient user and the conversational AI agent. In certain embodiments, clinical note 1000 may comprise a third generative text section 1006 comprising a data table including data related to the history of present illness for the patient user. For example, third generative text section 1006 may comprise data related to the management of diabetes in the patient user including, for example, medication log data, fasting blood glucose data and pre-meal blood glucose data. In certain embodiments, clinical note 1000 may comprise a fourth generative text section 1008 comprising a data table including a medical history for the patient user and one or more ICD-10 codes for clinical diagnosis and/or billing. In certain embodiments, clinical note 1000 may comprise a fifth generative text section 1010 comprising a generative text output that details a list of the current outpatient medications for the patient user. In certain embodiments, clinical note 1000 may comprise a sixth generative text section 1012 comprising a generative text output that details a list of known allergies for the patient user. In certain embodiments, clinical note 1000 may comprise a seventh generative text section 1014 comprising a data table that details a social history for the patient user. In certain embodiments, clinical note 1000 may comprise an eighth generative text section 1016 comprising a data table that details objective data for the patient user including, for example, biological data for the patient user (e.g., blood glucose data) and physiological data for the patient user (e.g., estimated body mass data). In certain embodiments, clinical note 1000 may comprise a ninth generative text section 1018 comprising an AI-generated assessment and treatment plan for the patient user. In certain embodiments, clinical note 1000 may comprise a tenth generative text section 1020 comprising AI-generated summary of the remote patient intervention according to the clinical protocol. In certain embodiments, clinical note 1000 may comprise an eleventh generative text section 1022 comprising AI-generated summary of the patient interaction with the clinical AI agent.

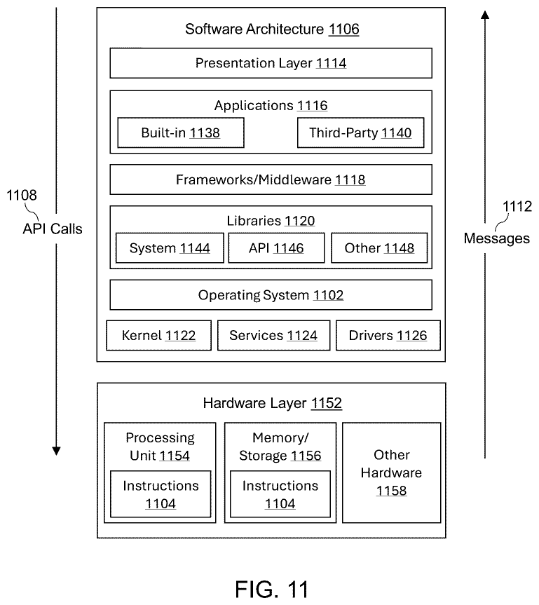

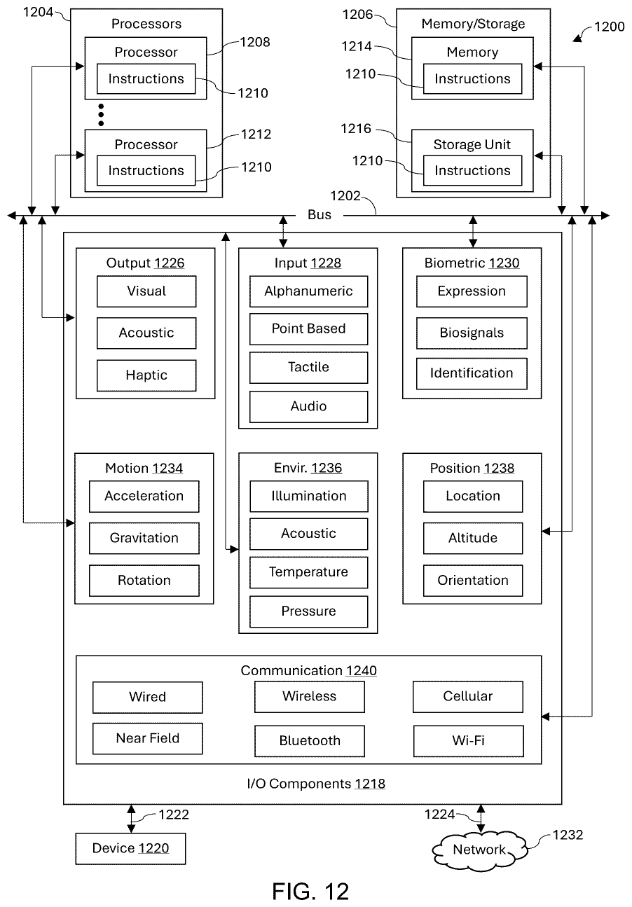

FIG. 11 is a block diagram illustrating an exemplary software architecture 1106, which may be used in conjunction with various hardware architectures herein described. FIG. 11 is a non-limiting example of a software architecture, and it will be appreciated that many other architectures may be implemented to facilitate the functionality described herein. The software architecture 1106 may execute on hardware such as machine 1200 of FIG. 12 that includes, among other things, processors 1204, memory 1214, and I/O components 1218. A representative hardware layer 1152 is illustrated and can represent, for example, the machine 1200 of FIG. 12. The representative hardware layer 1152 includes a processing unit 1154 having associated executable instructions 1104. Executable instructions 1104 represent the executable instructions of the software architecture 1106, including implementation of the methods, components and so forth described herein. The hardware layer 1152 also includes memory or storage modules memory/storage 1156, which also have executable instructions 1104. The hardware layer 1152 may also comprise other hardware 1158.

As used herein, the term “component” may refer to a device, physical entity or logic having boundaries defined by function or subroutine calls, branch points, application program interfaces (APIs), or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions.

Components may constitute either software components (e.g., code embodied on a machine-readable medium) or hardware components. A “hardware component” is a tangible unit capable of performing certain operations and may be configured or arranged in a certain physical manner. In various exemplary embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations.

A hardware component may be a special-purpose processor, such as a Field-Programmable Gate Array (FPGA) or an Application Specific Integrated Circuit (ASIC). A hardware component may also include programmable logic or circuitry that is temporarily configured by software to perform certain operations. For example, a hardware component may include software executed by a processor or other programmable processor. Once configured by such software, hardware components become specific machines (or specific components of a machine) uniquely tailored to perform the configured functions and are no longer processors. It will be appreciated that the decision to implement a hardware component mechanically, in dedicated and permanently configured circuitry, or in temporarily configured circuitry (e.g., configured by software) may be driven by cost and time considerations.

A processor may be, or include, any circuit, circuitry, or virtual circuit (a physical circuit emulated by logic executing on an actual processor) that manipulates data values according to control signals (e.g., “commands”, “op codes”, “machine code”, etc.) and which produces corresponding output signals that are applied to operate a machine. A processor may, for example, be a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC) or any combination thereof. A processor may further be a multi-core processor having two or more independent processors (sometimes referred to as “cores”) that may execute instructions contemporaneously. The processor as used herein may be a hardware component, which is in at least one of the devices, systems, servers and the like. The processor may include multiple cores and may be spread across multiple devices. The processor includes circuitry to execute instructions relating to the methods and structures described herein for determining relationships and outputting relationship data that is used by various device and their users.

Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a processor configured by software to become a special-purpose processor, the processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access.

For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components.

Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)). The performance of certain of the operations may be distributed among the processors, not only residing within a single machine, but deployed across a number of machines. In some exemplary embodiments, the processors or processor-implemented components may be located in a single geographic location (e.g., within a home environment, an office environment, or a server farm). In other exemplary embodiments, the processors or processor-implemented components may be distributed across a number of geographic locations.

In the exemplary architecture of FIG. 11, the software architecture 1106 may be conceptualized as a stack of layers where each layer provides particular functionality. For example, the software architecture 1106 may include layers such as an operating system 1102, libraries 1120, applications 1116 and a presentation layer 1114. Operationally, the applications 1116 or other components within the layers may invoke application programming interface (API) API calls 1108 through the software stack and receive messages 1112 in response to the API calls 1108. The layers illustrated are representative in nature and not all software architectures have all layers. For example, some mobile or special purpose operating systems may not provide a frameworks/middleware 1118, while others may provide such a layer. Other software architectures may include additional or different layers.

The operating system 1102 may manage hardware resources and provide common services. The operating system 1102 may include, for example, a kernel 1122, services 1124 and drivers 1126. The kernel 1122 may act as an abstraction layer between the hardware and the other software layers. For example, the kernel 1122 may be responsible for memory management, processor management (e.g., scheduling), component management, networking, security settings, and so on. The services 1124 may provide other common services for the other software layers. The drivers 1126 are responsible for controlling or interfacing with the underlying hardware. For instance, the drivers 1126 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 1120 provide a common infrastructure that is used by the applications 1116 or other components or layers. The libraries 1120 provide functionality that allows other software components to perform tasks in an easier fashion than to interface directly with the underlying operating system 1102 functionality (e.g., kernel 1122, services 1124 or drivers 1126). The libraries 1120 may include system libraries 1144 (e.g., C standard library) that may provide functions such as memory allocation functions, string manipulation functions, mathematical functions, and the like. In addition, the libraries 1120 may include API libraries 1146 such as media libraries (e.g., libraries to support presentation and manipulation of various media format such as MPREG4, H.264, MP3, AAC, AMR, JPG, PNG), graphics libraries (e.g., an OpenGL framework that may be used to render 2D and 3D in a graphic content on a display), database libraries (e.g., SQLite that may provide various relational database functions), web libraries (e.g., WebKit that may provide web browsing functionality), and the like. The libraries 1120 may also include a wide variety of other libraries 1148 to provide many other APIs to the applications 1116 and other software components/modules.

The frameworks/middleware 1118 (also sometimes referred to as middleware) provide a higher-level common infrastructure that may be used by the applications 1116 or other software components/modules. For example, the frameworks/middleware 1118 may provide various graphic user interface (GUI) functions, high-level resource management, high-level location services, and so forth. The frameworks/middleware 1118 may provide a broad spectrum of other APIs that may be utilized by the applications 1116 or other software components/modules, some of which may be specific to a particular operating system 1102 or platform.

The applications 1116 include built-in applications 1138 or third-party applications 1140. The third-party applications 1140 may invoke the API calls 1108 provided by the operating system 1102 to facilitate functionality described herein.

The applications 1116 may use built in operating system functions (e.g., kernel 1122, services 1124 or drivers 1126), libraries 1120, and frameworks/middleware 1118 to create user interfaces to interact with users of the system. Alternatively, or additionally, in some systems interactions with a user may occur through a presentation layer, such as presentation layer 1114. In these systems, the application/component “logic” can be separated from the aspects of the application/component that interact with a user.

FIG. 12 is a block diagram illustrating components (also referred to herein as “modules”) of a machine 1200, according to some exemplary embodiments, able to read instructions from a machine-readable medium (e.g., a machine-readable storage medium) and perform any one or more of the methodologies discussed herein. Specifically, FIG. 12 shows a diagrammatic representation of the machine 1200 in the example form of a computer system, within which instructions 1210 (e.g., software, a program, an application, an applet, an app, or other executable code) for causing the machine 1200 to perform any one or more of the methodologies discussed herein may be executed. As such, the instructions 1210 may be used to implement modules or components described herein. The instructions 1210 transform the non-programmed machine 1200 into a particular machine 1200 programmed to carry out the described and illustrated functions in the manner described. In alternative embodiments, the machine 1200 operates as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a laptop computer, a network router, a network switch, a network bridge, or any machine capable of executing the instructions 1210, sequentially or otherwise, that specify actions to be taken by machine 1200. Further, while only a single machine 1200 is illustrated, the term “machine” shall also be taken to include a collection of machines that individually or jointly execute the instructions 1210 to perform any one or more of the methodologies discussed herein.

The machine 1200 may include processors 1204, memory memory/storage 1206, and I/O components 1218, which may be configured to communicate with each other such as via a bus 1202. The memory/storage 1206 may include a memory 1214, such as a main memory, or other memory storage, and a storage unit 1216, both accessible to the processors 1204 such as via the bus 1202. The storage unit 1216 and memory 1214 store the instructions 1210 embodying any one or more of the methodologies or functions described herein. The instructions 1210 may also reside, completely or partially, within the memory 1214, within the storage unit 1216, within at least one of the processors 1204 (e.g., within the processor's cache memory), or any suitable combination thereof, during execution thereof by the machine 1200. Accordingly, the memory 1214, the storage unit 1216, and the memory of processors 1204 are examples of machine-readable media.

As used herein, the term “machine-readable medium,” “computer-readable medium,” or the like may refer to any component, device or other tangible media able to store instructions and data temporarily or permanently. Examples of such media may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) or any suitable combination thereof. The term “machine-readable medium” should be taken to include a single medium or multiple media (e.g., a centralized or distributed database, or associated caches and servers) able to store instructions. The term “machine-readable medium” may also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” may refer to a single storage apparatus or device, as well as “cloud-based” storage systems or storage networks that include multiple storage apparatus or devices. The term “machine-readable medium” excludes signals per se.

The I/O components 1218 may include a wide variety of components to provide a user interface for receiving input, providing output, producing output, transmitting information, exchanging information, capturing measurements, and so on. The specific I/O components 1218 that are included in the user interface of a particular machine 1200 will depend on the type of machine. It will be appreciated that the I/O components 1218 may include many other components that are not shown in FIG. 12. The I/O components 1218 are grouped according to functionality merely for simplifying the following discussion and the grouping is in no way limiting. In various exemplary embodiments, the I/O components 1218 may include output components 1226 and input components 1228. The output components 1226 may include visual components (e.g., a display such as a plasma display panel (PDP), a light emitting diode (LED) display, a liquid crystal display (LCD), a projector, or a cathode ray tube (CRT)), acoustic components (e.g., speakers), other signal generators, and so forth. The input components 1228 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like. The input components 1228 may also include one or more image-capturing devices, such as a digital camera for generating digital images or video.

In further exemplary embodiments, the I/O components 1218 may include biometric components 1230, motion components 1234, environmental environment components 1236, or position components 1238, as well as a wide array of other components. One or more of such components (or portions thereof) may collectively be referred to herein as a “sensor component” or “sensor” for collecting various data related to the machine 1200, the environment of the machine 1200, a user of the machine 1200, or a combinations thereof.

Communication may be implemented using a wide variety of technologies. The I/O components 1218 may include communication components 1240 operable to couple the machine 1200 to a network 1232 or devices 1220 via coupling 1222 and coupling 1224 respectively. For example, the communication components 1240 may include a network interface component or other suitable device to interface with the network 1232. In further examples, communication components 1240 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1220 may be another machine or any of a wide variety of peripheral devices (e.g., a peripheral device coupled via a Universal Serial Bus (USB)). Moreover, the communication components 1240 may detect identifiers or include components operable to detect identifiers.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

Any publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

Where a phrase similar to “at least one of A, B, or C,” “at least one of A, B, and C,” “one or more A, B, or C,” or “one or more of A, B, and C” is used, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that phases of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While certain exemplary embodiments have been described and shown in the accompanying drawings, it is to be understood that such embodiments are merely illustrative of, and not restrictive on, the broad invention, and that this invention is not limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1. A method for automatically formatting medical data for remote patient interventions, the method comprising:
configuring, with at least one server, a remote intervention protocol for a patient;
configuring, with the at least one server, a generative artificial intelligence (AI) model according to one or more parameters of the remote intervention protocol for the patient;
instantiating, with the at least one server, a conversational interaction between a conversational AI agent and a first client device associated with the patient, wherein the first client device comprises an audio input/output means;
outputting, with the conversational AI agent, one or more generative prompts according to the generative AI model at the first client device;
receiving, with the at least one server, one or more conversational responses from the patient via the first client device;
converting, with the at least one server, the one or more conversational responses from an audio data format to a text data format, wherein the text data format comprises a transcript of the conversational interaction between the conversational AI agent and the patient;
generating, with the at least one server, a formatted dataset from the transcript of the conversational interaction and one or more electronic medical records for the patient according to a data processing framework configured according to the remote intervention protocol,
wherein generating the formatted dataset comprises data cleaning, text normalization, tokenization, feature scaling, vectorization, and feature extraction;
processing, with the generative AI model, the formatted dataset to generate a clinical note for the patient,
wherein the clinical note documents at least a current stage of the remote intervention protocol, a present diagnosis, care provided during the conversational interaction, and at least one of a medical-necessity determination or a level-of-service determination for the remote patient intervention;
storing, in a non-transitory storage device communicably engaged with the at least one server, the clinical note for the patient in a standardized format;
providing, with the at least one server, a notification to a second client device associated with a provider user in response to storing the clinical note; and
rendering, with the at least one server, the clinical note in the standardized format at the second client device.

2. The method of claim 1 further comprising receiving, at a user interface of the second client device, an electronic signature for the provider user in response to rendering the clinical note.

3. The method of claim 2 further comprising applying, with the at least one server, the electronic signature for the provider user to the clinical note and storing the signed clinical note in the standardized format, wherein applying the electronic signature comprises applying a timestamped electronic signature to the clinical note.

4. The method of claim 3 further comprising, after applying the timestamped electronic signature, hashing or encrypting the signed clinical note to secure the clinical note and the electronic signature.

5. The method of claim 4 further comprising recording metadata for the electronic signature, wherein the metadata comprises at least one of a timestamp, a geolocation, an Internet Protocol address, or practitioner user interaction data.

6. The method of claim 1 further comprising initiating, with the at least one server, at least one routine of the remote intervention protocol in response to at least one user-generated input by the patient at the first client device, wherein the at least one routine corresponds to a protocol state of the remote intervention protocol.

7. The method of claim 1 further comprising determining, with the generative AI model, one or more medical billing codes for the conversational interaction by extracting and analyzing one or more features from the formatted dataset.

8. The method of claim 7 further comprising assigning, with the at least one server, the one or more determined medical billing codes for the conversational interaction to the clinical note according to the standardized format and assigning the one or more determined medical billing codes to the formatted dataset.

9. The method of claim 1 further comprising receiving, via the second client device, one or more user-generated inputs from the provider user, wherein the one or more user-generated inputs comprise one or more additions or revisions to the clinical note.

10. The method of claim 9 further comprising updating, with the at least one server, the clinical note according to the one or more user-generated inputs from the provider user and storing the updated clinical note in the non-transitory storage device, wherein updating the clinical note comprises updating at least one of the formatted dataset, one or more extracted features, or one or more medical billing codes according to the one or more additions or revisions.

11. A system automatically formatting medical data for remote patient interventions, the system comprising:
a first client device associated with a patient user, the first client device comprising an audio input/output means;
a second client device associated with a practitioner user, the second client device comprising a user interface; and
at least one server communicably engaged with the first client device and the second client device via a network communications interface, the at least one server comprising at least one processor and a non-transitory computer-readable storage device communicably engaged with the at least one processor, the non-transitory computer-readable storage device comprising processor-executable instructions stored thereon that, when executed, cause the at least one processor to perform one or more operations, the one or more operations comprising:
configuring a remote intervention protocol for the patient user;
configuring a generative artificial intelligence (AI) model according to one or more parameters of the remote intervention protocol for the patient user;
instantiating a conversational interaction between a conversational AI agent and the first client device;
outputting one or more generative prompts according to the generative AI model at the first client device;
receiving one or more conversational responses from the patient via the first client device;
converting the one or more conversational responses from an audio data format to a text data format, wherein the text data format comprises a transcript of the conversational interaction between the conversational AI agent and the patient user;
generating a formatted dataset from the transcript of the conversational interaction and one or more electronic medical records for the patient user according to a data processing framework configured according to the remote intervention protocol,
wherein generating the formatted dataset comprises data cleaning, text normalization, tokenization, feature scaling, vectorization, and feature extraction;
processing, with the generative AI model, the formatted dataset to generate a clinical note for the patient user,
wherein the clinical note documents at least a current stage of the remote intervention protocol, a present diagnosis, care provided during the conversational interaction, and at least one of a medical-necessity determination or a level-of-service determination for the remote patient intervention;
storing, in the non-transitory computer-readable storage device, the clinical note for the patient user in a standardized format;
providing, via the network communications interface, a notification to the second client device in response to storing the clinical note; and
rendering the clinical note in the standardized format at the second client device.

12. The system of claim 11 wherein the one or more operations further comprise receiving, via the second client device, an electronic signature from the provider user in response to rendering the clinical note at the second client device.

13. The system of claim 12 wherein the one or more operations further comprise applying the electronic signature for the provider user to the clinical note and storing the signed clinical note in the standardized format, wherein applying the electronic signature comprises applying a timestamped electronic signature to the clinical note.

14. The system of claim 13 wherein the one or more operations further comprise, after applying the timestamped electronic signature, hashing or encrypting the signed clinical note to secure the clinical note and the electronic signature.

15. The system of claim 14 wherein the one or more operations further comprise recording metadata for the electronic signature, wherein the metadata comprises at least one of a timestamp, a geolocation, an Internet Protocol address, or practitioner user interaction data.

16. The system of claim 11 wherein the one or more operations further comprise determining, with the generative AI model, one or more medical billing codes for the conversational interaction by extracting and analyzing one or more features from the formatted dataset.

17. The system of claim 16 wherein the one or more operations further comprise assigning the one or more determined medical billing codes for the conversational interaction to the clinical note according to the standardized format and assigning the one or more determined medical billing codes to the formatted dataset.

18. The system of claim 11 wherein the one or more operations further comprise receiving, via the second client device, one or more user-generated inputs from the provider user, wherein the one or more user-generated inputs comprise one or more additions or revisions to the clinical note.

19. The system of claim 18 wherein the one or more operations further comprise updating the clinical note according to the one or more user-generated inputs from the provider user and storing the updated clinical note in the non-transitory storage device, wherein updating the clinical note comprises updating at least one of the formatted dataset, one or more extracted features, or one or more medical billing codes according to the one or more additions or revisions.

20. A non-transitory computer readable medium having processor-executable instructions stored thereon that, when executed by at least one processor, are configured to cause the at least one processor to execute one or more operations of a method for automatically formatting medical data for remote patient interventions, the one or more operations comprising:
configuring a remote intervention protocol for the patient user;
configuring a generative artificial intelligence (AI) model according to one or more parameters of the remote intervention protocol for the patient user;
instantiating a conversational interaction between a conversational AI agent and a first client device associated with the patient user;
outputting one or more generative prompts according to the generative AI model at the first client device;
receiving one or more conversational responses from the patient via the first client device;
converting the one or more conversational responses from an audio data format to a text data format, wherein the text data format comprises a transcript of the conversational interaction between the conversational AI agent and the patient user;
generating a formatted dataset from the transcript of the conversational interaction and one or more electronic medical records for the patient user according to a data processing framework configured according to the remote intervention protocol,
wherein generating the formatted dataset comprises data cleaning, text normalization, tokenization, feature scaling, vectorization, and feature extraction;
processing, with the generative AI model, the formatted dataset to generate a clinical note for the patient user,
wherein the clinical note documents at least a current stage of the remote intervention protocol, a present diagnosis, care provided during the conversational interaction, and at least one of a medical-necessity determination or a level-of-service determination for the remote patient intervention;
storing, in the non-transitory computer-readable storage device, the clinical note for the patient user in a standardized format;
providing, via the network communications interface, a notification to a second client device associated with a provider user in response to storing the clinical note; and
rendering the clinical note in the standardized format at the second client device.
